(19) United States
(12) Patent Application Publication
(10) Pub. No.: US 2009/0311172 A1
(43) Pub. Date: Dec. 17, 2009

(54) Massage cream and massaging method using the same

(75) Inventor: Takao Saruta, Tokyo (JP)

Correspondence Address: SQUIRE, SANDERS & DEMPSEY L.L.P., 8000 TOWERS CRESCENT DRIVE, 14TH FLOOR, VIENNA, VA, 22182-6212, US

(73) Assignee: SUEAM Corporation

(21) Appl. No.: 12/453,767

(22) Filed: May 21, 2009

Related U.S. Application Data
(60) Provisional application No. 61/129,306, filed on Jun. 17, 2008.

(51) Int. Cl. A61K 51/00

(52) U.S. Cl. 424/1.11

(57) ABSTRACT

The present invention relates to a massage cream which contains 5.0 Bq to 300.0 Bq of powders of one or more radioactive materials per 1 g of base materials. Furthermore in the massage cream the radioactive materials include 4.0 Bq to 240.0 Bq of thorium. Moreover in the massage cream the radioactive materials further include 1.0 Bq to 60.0 Bq of uranium. With the massage cream, medical effects can be given to the body of a person.

### CROSS-REFERENCE TO RELATED APPLICATION:

This application claims the benefit of U.S. Provisional Application No. 61/129,306 filed on Jun. 17, 2008, which is hereby incorporated by reference.

### BACKGROUND OF THE INVENTION

1. Field of the Invention

Generally the present invention relates to massage creams. More specifically, the present invention relates to a massage cream that is capable of providing medical effects.

2. Description of the Related Art

Massage is one of the oldest, simplest forms of therapy and is a system of kneading and rubbing the various parts of the body by using hands, fingers, elbows and feet. Massage works on the soft tissues (muscles, tendons, ligaments, etc.) directly to increase the blood and lymph circulations and to improve the elimination of waste products throughout the body.

It appears that the origin of massage is French manual therapy. However, current massage can be classified into many different styles, including lomi-lomi, shiatsu, Thai and Swedish.

The main benefits of massage are the relief of pain and the relaxing, stimulating and tuning of the body. However, the purpose of massage is not only limited to health care but also has beauty or diet benefits.

A massage cream can be used to conduct massage with (see JPH10-087430). Many types of massage creams are available. Such massage creams aim at facilitating massage operations and enhancing the effect of massage. Actually a massage cream can reduce the friction between the skin and the fingers, thereby preventing excessive tension from being applied to the skin. Also a massage cream acts on the muscle directly to thereby help with the relief of the muscle stress, the increase in the lymph circulation, and the elimination of the waste products.

The hardness and viscosity of a massage cream may be varied depending on the style or purpose of the massage or the body parts to which massage is given. This enables a massage cream to be presented in different forms, including oil and jelly forms.

A massage cream may contain, in addition to the main components, some extra components that have aroma, moisturizer or coolant agents.

However the precise effectiveness of massage creams is not proved in terms of medical aspects. Therefore a massage cream that provides actual medical effects has been in demand. Taking the above description into account, the present invention has been conceived. A goal of the present invention is to create a massage cream that is capable of performing specific medical effects.

### SUMMARY OF THE INVENTION

According to an aspect of the present invention, there is provided, a massage cream containing 5.0 Bq to 300.0 Bq of powders of one or more radioactive materials per 1 g of base materials.

According to another aspect of the present invention, there is provided, the massage cream in which the radioactive materials include 4.0 Bq to 240.0 Bq of thorium.

According to still another aspect of the present invention, there is provided, the massage cream in which the radioactive materials further include 1.0 Bq to 60.0 Bq of uranium.

Other aspects, features and advantages of the present invention will become apparent upon reading the following specification and claims when taken in conjunction with the accompanying drawings.

### DETAILED DESCRIPTION OF THE EXEMPLARY EMBODIMENTS OF THE INVENTION

A description will be given below in detail, of a massage cream of one embodiment according to the present invention.

A massage cream of this embodiment has developed in order to give “Hormesis effect” to a client easily.

The “Hormesis” is named by German scientist Dr. Hugo Schulz in the early 1900's, and it means that certain products may damage human lives by a large amount but stimulate human bodies excellently by a small amount.

As a result of accurate study, the inventor, et al. have successfully achieved the Hormesis effect in massage. Specifically they create a massage cream by adding a predetermined amount of low-level radioactive materials to base materials of a massage cream. Then, the created massage cream is applied to the body of a subject during the massage.

Therefore a massage cream of one embodiment according to the present invention contains fine powders of one or more radioactive materials by a predetermined amount.

Base materials of the massage cream may be ones typically used for a massage cream, and the viscosity of the cream is adjusted in accordance of the body parts to which massage is to be given.

The radioactive materials are not limited to specific materials, but their particle diameters need to be 0.1 μm to 2.0 μm, preferably 0.3 μm to 0.8 μm in consideration of the skin absorption of the radioactive material.

Examples of the radioactive materials include a mineral ore containing radioactive element, such as Thorium (monazite), Uranium, Radium, Radon, Polonium or Bismuth.

Such radioactive materials can be added to the base materials of a massage cream during, before or after the production of the massage cream. Alternatively the radioactive materials may be added to a massage cream that has been already created. Following this the massage cream is stirred sufficiently.

The amounts of the radioactive materials can be varied depending on the style of massage and other conditions, but preferably the amounts are 5.0 Bq to 300.0 Bq per 1 g of the base materials. Moreover, the radioactive materials may include 4.0 Bq to 240.0 Bq of Th. Also, the radioactive materials may include 1.0 Bq to 60.0 Bq of U.

The upper limit at which a massage cream contains the radioactive materials is not limited to a specific value. However the current regulation confines the use of a radioactive material of more than 370 Bq/g. In consideration of this fact the upper limit is set 300 Bq/g in this embodiment.

The above massage cream may contain various additives unless they inhibit the effect of the radioactive material. Preferably the additives are ones described in Japanese Standards of Cosmetic Ingredients (JSCI). Examples of the additives include, but not limited to, aroma chemicals, antioxidants, herbal materials such as daylily, herbal extract, vegetable origin activation, and squalene.

Alternatively the massage cream may be produced by preparing dispersion liquid containing fine powders of the radioactive materials and by adding the dispersion liquid to the base materials. This is how the massage cream can be prepared prior to massage.

The massage cream produced in the above way makes it possible to stimulate the body of a person appropriately and to give it the Hormesis effect.

The inventor, et al. conducted an experiment how the massage cream influences the bodies of humans. This result is as follows.

TABLE 1

| ALLEVIATION AND HEALING | NUMBER | RATIO(%) |
|---|---|---|
| pain in shoulders, waist and knees | 91 | 23.7 |
| headache, toothache and stomatitis | 21 | 5.5 |
| muscle pain, fatigue, swelling and lump | 62 | 16.0 |
| bruise and sprained thumb | 13 | 3.4 |
| cold constitution | 13 | 3.4 |
| fleck and wrinkles on face and dry skin | 40 | 10.4 |
| diet | 11 | |
| dermatitis, scar and athlete's foot | 28 | 7.3 |
| pimple, verruca and hump | 11 | |
| constipation and body scent | 17 | 4.4 |
| flu, bronchial infection, pollen allergen and | 20 | 5.2 |
| rhinitis | | |
| trouble of brain, blood pressure and heart | 18 | 4.7 |
| trouble of inward organs and glycaemia | 15 | 3.9 |
| insomnia, stress and depression | 13 | 3.4 |
| alopecia | 2 | |

The above result shows that the massage using the massage cream enables cellular repair, cellular stimulant and the improvement of hormone balance.

From the aforementioned explanation, those skilled in the art ascertain the essential characteristics of the present invention and can make the various modifications and variations to the present invention to adapt it to various usages and conditions without departing from the spirit and scope of the claims.

We claim:

1. A massage cream containing 5.0 Bq to 300.0 Bq of powders of one or more radioactive materials per 1 g of base materials.

2. The massage cream according to claim 1 wherein the radioactive materials include 4.0 Bq to 240.0 Bq of thorium.

3. The massage cream according to claim 2 wherein the radioactive materials further include 1.0 Bq to 60.0 Bq of uranium.
